(19) United States
(12) Patent Application Publication
(10) Pub. No.: US 2018/0000761 A1
(43) Pub. Date: Jan. 4, 2018

(54) Methods for Making Catechin Derivatives

(71) Applicant: UNIVERSITY OF SOUTH FLORIDA, Tampa, FL (US)

(72) Inventors: James William Leahy, Lutz, FL (US); Andrea A. Lemus, Tampa, FL (US)

(73) Assignee: UNIVERSITY OF SOUTH FLORIDA, Tampa, FL (US)

(21) Appl. No.: 15/546,601

(22) PCT Filed: Dec. 12, 2016
(86) PCT No.: PCT/US16/66143

§ 371 Date: Jul. 26, 2017

Related U.S. Application Data
(60) Provisional application No. 62/265,707, filed on Dec. 10, 2015.
(60) Provisional application No. 62/374,173, filed on Aug. 12, 2016.

(51) Int. Cl. A61K 31/192; A61K 31/685; C07D 307/33; A61K 31/352; C07D 493/04

(52) U.S. Cl. CPC A61K 31/192; C07D 307/33; A61K 31/352; A61K 31/685; C07D 493/04

(57) ABSTRACT

Disclosed herein are methods for making a catechin derivative, or a salt thereof. The methods can include: contacting an alcohol protected aromatic aldehyde and a triphenyl phosphonium ylide to make an alcohol protected aromatic olefin; hydrogenating the alcohol protected aromatic olefin to make alcohol protected aromatic compound; and deprotecting the alcohol protected aromatic compound to make the catechin derivative.

### CROSS-REFERENCE TO RELATED APPLICATIONS

This application claims the benefit of U.S. Provisional Application Ser. No. 62/265,707, filed Dec. 10, 2015 and 62/374,173, filed Aug. 12, 2016, the disclosures of which are hereby incorporated by reference in their entirety, including all figures, tables and drawings.

### BACKGROUND OF THE INVENTION

Catechin is a secondary metabolite that can be found in a wide variety of plants. The molecular structure of catechin is show below:

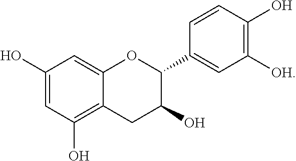 [SMILES: Oc1cc(O)c2c(c1)O[C@H](c1ccc(O)c(O)c1)[C@@H](O)C2]

Catechin belongs to the flavonoid class of antioxidant molecules, and has recently garnered interest in the treatment of diabetes and heart disease [1, 2]. For example, catechin can have cardioprotective properties through such pathways as vasodilation.

Catechin is metabolized by the liver and intestines. Catechin can be metabolized through methylation, ring cleavage, dehydroxylation, sulfonation, and other transformations, some of which are unique to certain organisms. While some of these metabolites can be extracted in sufficient quantity and purity to be sold as nutritional supplements, many of the minor metabolites are only available in such small quantities that their full evaluation cannot be determined. Indeed, it may even be that some of the beneficial biological properties are only due to a limited number of the metabolites.

### BRIEF SUMMARY OF THE INVENTION

Because of the lack of adequate isolated quantities of the metabolites and derivatives of catechin, there is a need to develop new synthetic methods for the synthesis of these compounds. Provided herein are methods for making the catechin derivatives, or salts thereof. In one specific embodiment, the method can include: contacting an alcohol protected aromatic aldehyde and a triphenyl phosphonium ylide to make an alcohol protected aromatic olefin; hydrogenating the alcohol protected aromatic olefin to make an alcohol protected aromatic compound; and deprotecting the alcohol protected aromatic compound to make the catechin derivative.

In another specific embodiment, the method can include:

contacting a

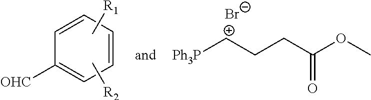

to make

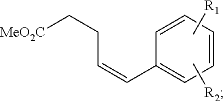

contacting

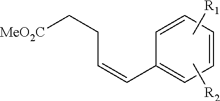

with a first base to make

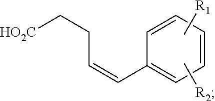

contacting

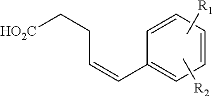

and N-bromosuccinimide to make

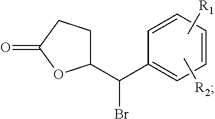

contacting

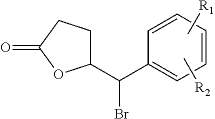

and a free radical initiator and a hydrogen donating compound to make

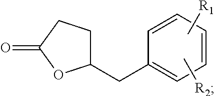

and contacting

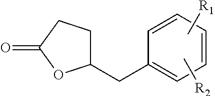

and a first hydrogenation catalyst to make the catechin derivative, where the catechin derivative has a formula

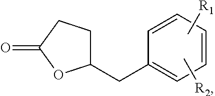

where R₁ and R₂ are independently selected from the group consisting of —H, —OH, —OCH₃, —OBn, —OSO₃H, —H, —OH, —OCH₃, —OSO₃H, tert-butyldimethylsilyl ether, trimethylsilyl, tert-butyldiphenylsilyl, triisopropylsilyl and alcohol protecting groups, such as methoxymethyl ether, t-Butyl ether, allyl ether, —OAc tetrahydropyranyl ether.

In another specific embodiment, the method can further include:

contacting a catechin derivative,

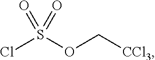

and a third base to make

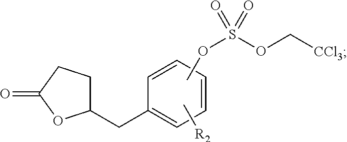

contacting

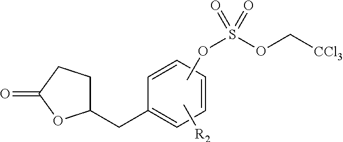

and a second hydrogenation catalyst to make

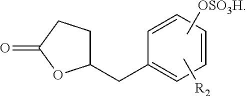

### BRIEF DESCRIPTION OF THE DRAWINGS

In the following detailed description, reference is made to the accompanying figures, depicting exemplary, non-limiting and non-exhaustive embodiments of the invention. So that the manner in which the above recited features of the present invention can be understood in detail, a more particular description of the invention, briefly summarized above, can be had by reference to the embodiments, some of which are illustrated in the appended figures. It should be noted, however, that the appended figures illustrate only typical embodiments of this invention and are therefore not to be considered limiting of its scope, for the invention can admit to other equally effective embodiments.

FIG. 1 is an embodiment of a method for making catechin derivatives.

FIG. 2 is an embodiment of a method for further making catechin derivatives.

FIG. 3 shows an example of the new synthetic method for the synthesis of catechin derivatives.

FIG. 4 shows generalized embodiments for methods further making catechin derivatives.

FIG. 5 shows generalized embodiments for methods further making catechin derivatives.

FIG. 6 shows the experimental reaction scheme for making compound M36 from a alcohol protected aromatic aldehyde, compound 1.

FIG. 7 are graphs that the effectiveness of catechin metabolites in reducing TNF-α in presence of LPS.

### DETAILED DISCLOSURE OF THE INVENTION

The method for making the catechin derivatives, or salts thereof, can include, but is not limited to: contacting an alcohol protected aromatic aldehyde and a triphenyl phosphonium ylide to make an alcohol protected aromatic olefin; hydrogenating the alcohol protected aromatic olefin to make an alcohol protected aromatic compound; and deprotecting the alcohol protected aromatic compound to make the catechin derivative.

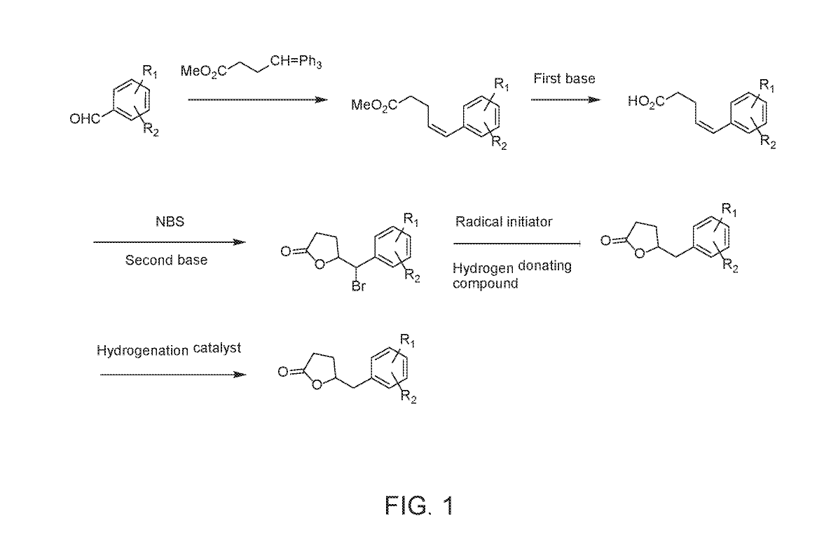

FIG. 1 is an embodiment of a method for making catechin derivatives. The contacting an alcohol protected aromatic aldehyde and a triphenyl phosphonium ylide to make an alcohol protected aromatic olefin can include:

contacting a

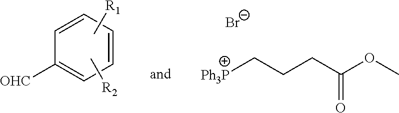

to make

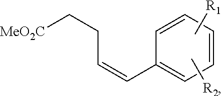

where R₁ and R₂ are independently selected from the group consisting of —H, —OH, —OCH₃, —OBn, —OSO₃H, and alcohol protecting groups, such as —OBn, methoxymethyl ether, t-Butyl ether, allyl ether, —OAc, and tetrahydropyranyl ether.

The alcohol protected aromatic aldehyde can include, but is not limited to: compounds of a formula:

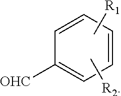

The triphenyl phosphonium ylide can include, but is not limited to: compounds of structure:

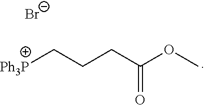

The alcohol protected aromatic olefin can include, but is not limited to: compounds of formula:

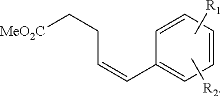

In FIG. 1, the hydrogenating the alcohol protected aromatic olefin to make an alcohol protected aromatic compound can include, but is not limited to:

contacting

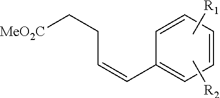

with a first base to make

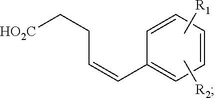

contacting

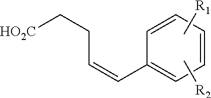

and N-bromosuccinimide to make

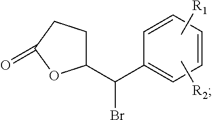

and contacting

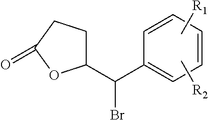

and a free radical initiator and a hydrogen donating compound to make

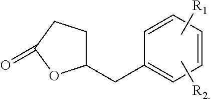

The alcohol protected aromatic olefin can include, but is not limited to, compounds of formula:

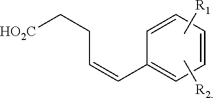

The alcohol protected aromatic compound can include, but is not limited to, compounds of formula:

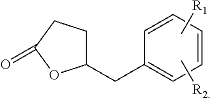

In FIG. 1, the deprotecting the alcohol protected alcohol compound to make the catechin derivative, can include, but is not limited to:

contacting

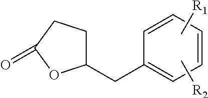

and a first hydrogenation catalyst to make the catechin derivative.

The catechin derivative can include but is not limited to, compounds with a formula:

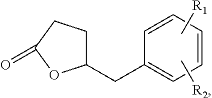

where R₁ and R₂ are independently selected from the group consisting of —H, —OH, —OCH₃, —OBn, —OSO₃H, and alcohol protecting groups, such as —OBn, methoxymethyl ether, t-Butyl ether, allyl ether, —OAc, and tetrahydropyranyl ether.

The first base can include, but is not limited to, sodium hydroxide, ammonium hydroxide, ammonium sulfate, lithium hydroxide, potassium hydroxide, triethylene tetraamine, diethylene triamine, triethylamine, urea, lithium diisopropylamide,

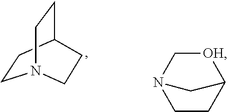

and mixtures thereof.

The free radical initiator can include, but is not limited to, peroxides, hydroperoxides, peresters, and azo compounds, and mixtures thereof. Examples of suitable free radical initiators can include, but are not limited to: dicumyl peroxide, di-t-butyl peroxide, t-butylperoxybenzoate, 2,5-dimethyl-2,5-di(t-butylperoxy)hexane, t-butyl peroxyneodecanoate, 2,5-dimethyl-2,5-di(t-butylperoxy)hexyne, t-amyl peroxypivalate, 1,3-bis(t-butylperoxyisopropyl)benzene, tert-amylperoxy 2-ethyl hexanoate, t-butylperoxy 2-ethyl hexanoate, t-butyl peroxy isobutyrate, t-butylperoxy isopropyl carbonate, t-butylperoxy 3,5,5-trimethylhexanoate, 2,5-dimethyl-2,5-di(benzolyperoxy)hexane, n-butyl 4,4-di(t-butylperoxy)valcratic, t-butylcumyl peroxide, di(2-t-butylperoxy isopropyl)benzene, t-butyl hydroperoxide, cumyl hydroperoxide and mixtures thereof. Examples of suitable azo compounds include 2,2′-azobisisopropionitrile, 2,2′-azobisisobutyronitrile (AIBN), dimethyl azoisobutyrate, 1,1′-azobis (cyclohexanecarbonitrile), 2,2′-azobis(2-methylpropane), and mixtures thereof.

The first hydrogenation catalyst can include, but is not limited to, platinum, palladium, such as Pd/C, rhodium, ruthenium and nickel.

The first hydrogen donating compound can include, but is not limited to, H₂, formic acid, isopropanol, and dihydroanthracene.

The contacting of

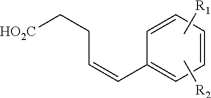

and N-bromosuccinimide to make

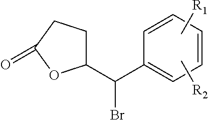

can further include contacting with PhCO₂H, a second base, and a chiral catalyst.

The second base can include, but is not limited to, sodium hydroxide, ammonium hydroxide, ammonium sulfate, lithium hydroxide, potassium hydroxide, triethylene tetraamine, diethylene triamine, triethylamine, urea, lithium diisopropylamide,

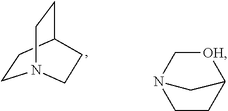

and mixtures thereof.

The chiral catalyst can include, but is not limited to, (DHQ)₂PHAL and (DHQD)₂PHAL. Using a chiral catalyst in the method can make the resulting catechin derivative enantiomerically pure.

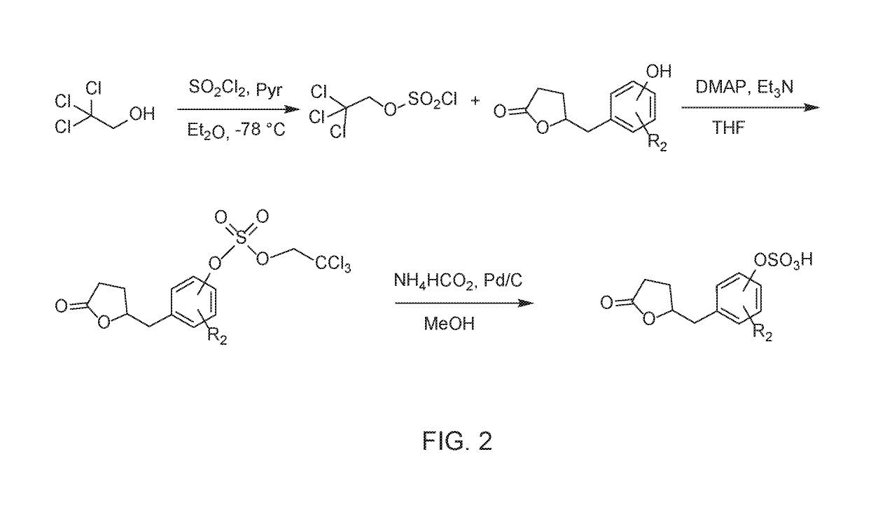

FIG. 2 is an embodiment of a method for further making catechin derivatives. The method can include, but is not limited to:

contacting the catechin derivative,

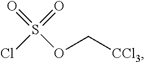

and a third base to make

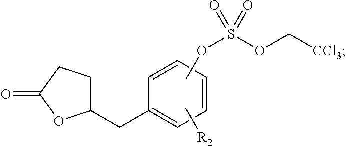

contacting

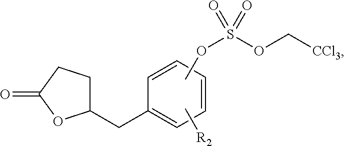

a second hydrogenation catalyst, and a second hydrogen donor compound to make a catechin derivative, where the catechin derivative can include but is not limited to, compounds of formula:

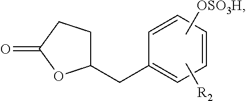

where R₂ is selected from —H, —OH, —OCH₃, —OBn, —OSO₃H, and alcohol protecting groups, such as —OBn, methoxymethyl ether, t-Butyl ether, allyl ether, —OAc, and tetrahydropyranyl ether.

The third base can include, but is not limited to, sodium hydroxide, ammonium hydroxide, ammonium sulfate, lithium hydroxide, potassium hydroxide, triethylene tetraamine, diethylene triamine, triethylamine, urea, lithium diisopropylamide,

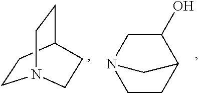

and mixtures thereof.

The second hydrogenation catalyst can include, but is not limited to, platinum, palladium, such as Pd/C, rhodium, ruthenium and nickel.

The second hydrogen donating compound can include, but is not limited to, H₂, formic acid, isopropanol, and dihydroanthracene.

Any of the reactions described herein can include one or more solvents. The solvents can include, water, alcohol, glycol, acetonitrile, dimethyl sulfoxide, N,N-dimethylformamide, N-methylpyrrolidone, and mixtures thereof.

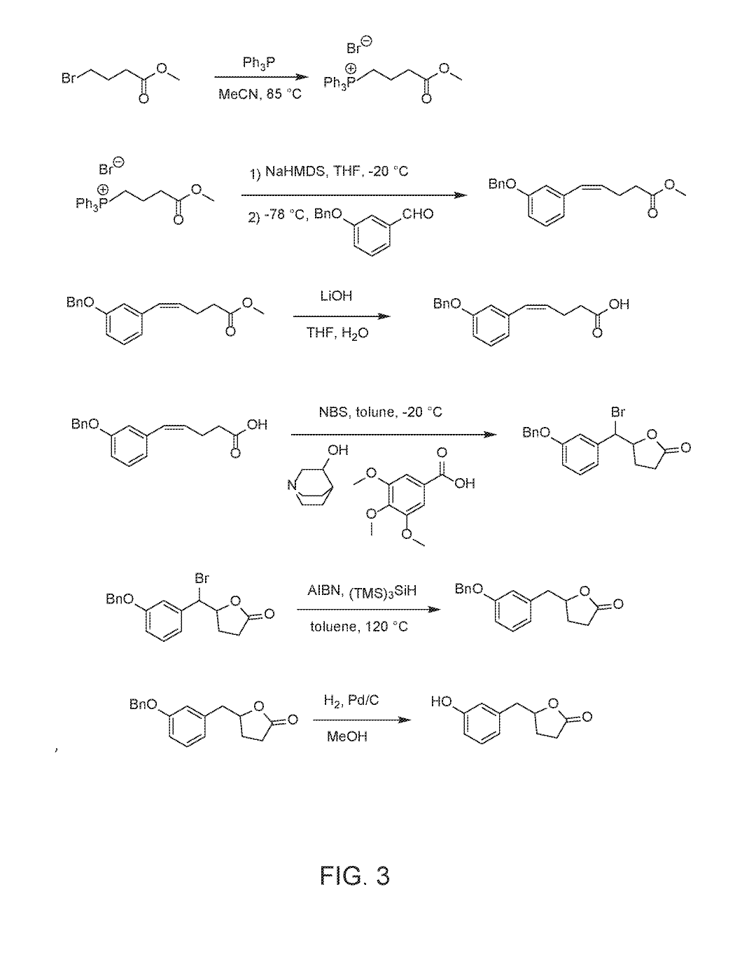

FIG. 3 shows an example of the synthetic method for the synthesis of catechin derivatives. The compound of the structure:

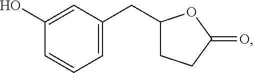 [SMILES: O=C1CCC(Cc2cccc(O)c2)O1]

is a major lactone metabolite present in rat urine analyses after consumption of catechin. This catechin metabolite can be further derivatized to a compound of the formula:

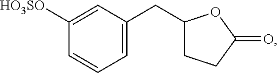

using the method show in FIG. 2.

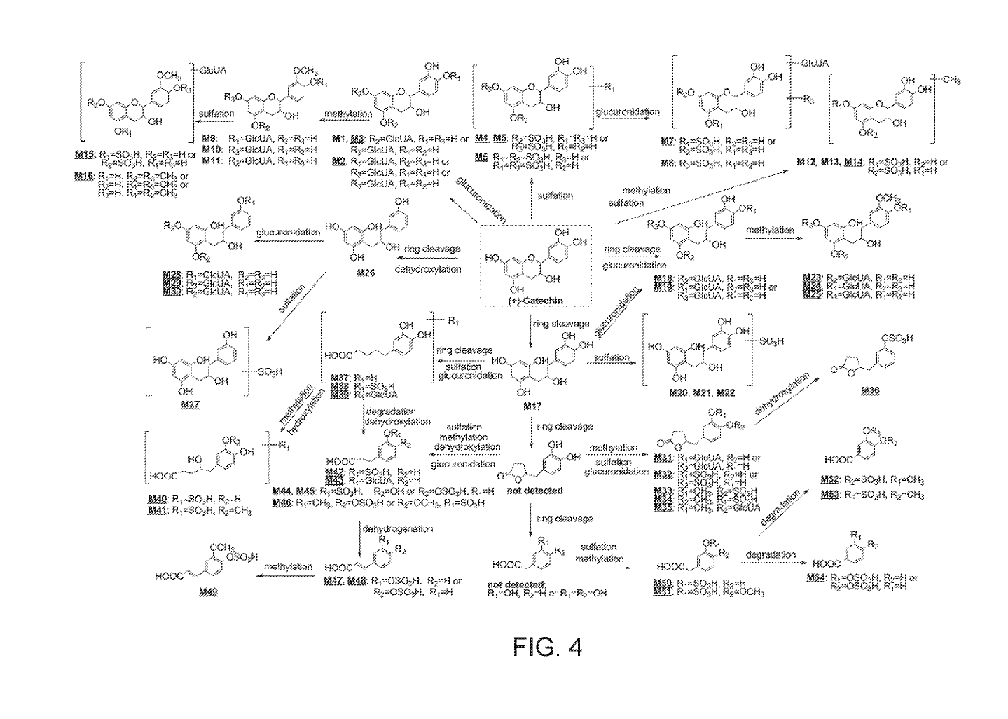

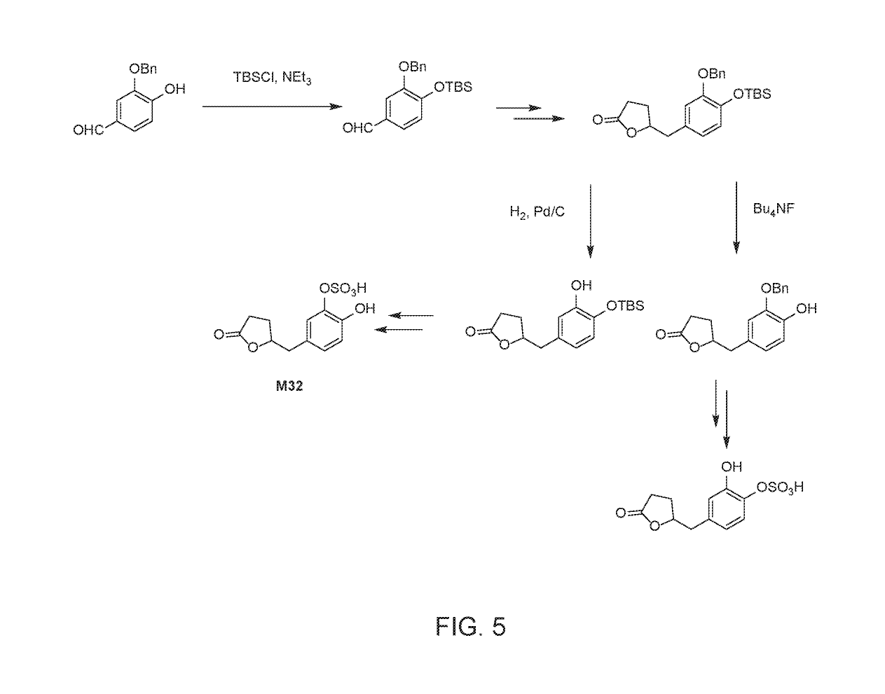

FIGS. 4 and 5 show how the new synthetic methods can be further modified to form other catechin derivatives in a similar manner. For example, in precursors possessing multiple alcohol moieties (—OH), and where the product requires modifying only one alcohol moiety to the corresponding sulfate, appropriate orthogonal protection scheme is used that allows one to be deprotected. Catechin derivatives, such as M32 can be prepared according to the new synthetic method from commercially available 1. Lactones can be formed from the alcohol protected aromatic aldehyde using condensation reaction, e.g., reacting with lithium diisopropylamide [3]. Moreover, since the oxygens can be selectively liberated as needed, it allows for the generation of M31, M33, M34 or M35. In fact, the availability of aldehyde intermediates also open the door for the preparation of many of the other metabolites as well. For example, they can be oxidized to the corresponding carboxylic acids, which allows for the synthesis of M52, M53 and M54. An Arndt-Eistert homologation leads to M50 and M51. Alternatively, a Knoevenagel condensation of the initial aldehydes allows for the synthesis of M47, M48 and M49.

With a starting alcohol protected aromatic aldehyde, carboxylic acids can be formed by oxidation of the aldehyde by treating the precursor with 4 mol equivalent of vanadyl acetylacetonate (VO(AcAc)₂) and hydrogen peroxide [4]. The intermediate is treated with acetonitrile at room temperature to form the carboxylic acid. Alternatively, treating the intermediate with a primary alcohol forms an ester. Carboxylic acids can also be formed from aromatic aldehydes of the invention by treating the aromatic aldehyde with sodium perborate [5].

### Materials and Methods

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

The catechin derivative M36, 3-[(5-oxotetrahydrofuran-2-yl)methyl]phenyl hydrogen sulfate, is show below:

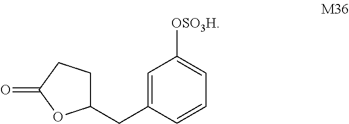

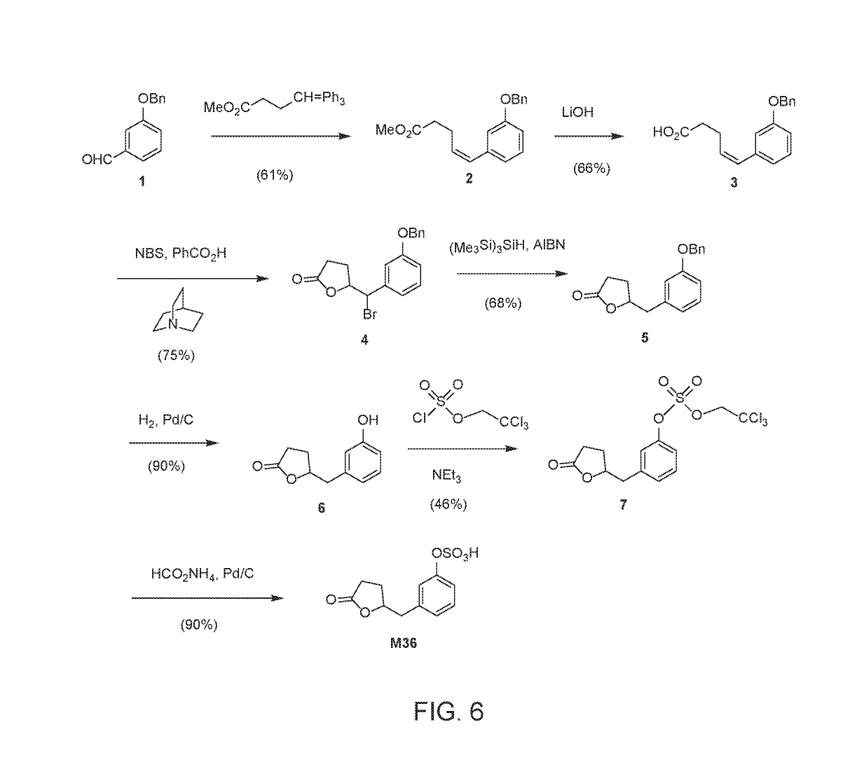

FIG. 6 shows the experimental reaction scheme for making compound M36 from a protected aromatic aldehyde, compound 1. Compound M36 can be synthesized from commercially available compound 1, a benzyl-protected aromatic aldehyde. Compound 1 can then be subjected to a Wittig olefination to give the 1-protected, 3-allylated compound 2. Compound 2 was saponified to form 3. N-bromo succinimide was used with quinuclidine to cyclize the allyl moiety to form a lactone 4, followed by deprotection of the phenyl and formation of the 3-substituted phenol 6. The phenol was sulfoxinated at the 1-hydroxy position via the trichloroethyl derivative to give 7, which allows for the chemical purification of the intermediate and facilitates its further manipulation to the final product, catechin metabolite M36.

### Synthesis of Compound 1

In a 500 mL round-bottomed flask was added 3-hydroxybenzaldehyde (3.00 g, 24.57 mmol), potassium carbonate (6.80 g, 49.2 mmol), and benzyl bromide (2.92 mL, 24.57 mmol) in acetonitrile (150 mL) to give a brown suspension that was left stirring overnight at room temperature. The solvent was then evaporated, and the beige residue was diluted with water and extracted with ethyl acetate (3×75 mL). The organic layer was dried with magnesium sulfate and concentrated on a rotary evaporator. Purification by column chromatography (5% ethyl acetate in hexanes) gave compound 1 as a white crystalline solid (5.0215 g, 23.66 mmol, 96%). ¹H NMR (400 MHz, CDCl₃) δ ppm 5.11 (s, 2H) 7.32-7.52 (m, 9H) 9.96 (s, 1H). m/z 213 [M+H]⁺.

### Synthesis of Compound 2

In a 500 mL round-bottomed flask was added methyl 4-bromobutanoate (14.70 g, 81 mmol) and triphenylphosphine (21.50 g, 82 mmol) in acetonitrile (130 mL), and the mixture was heated to 90° C. overnight to give a colorless suspension. The solution was removed from heat and the solvent was evaporated. The sludge was recrystallized from cold 1:1 acetonitrile and diethyl ether to give a total of 34.3 g (77 mmol, 95% yield) of (4-methoxy-4-oxobutyl)triphenylphosphonium bromide as white crystals. ¹H NMR (500 MHz, DMSO-d₆) δ ppm 1.69-1.81 (m, 2H) 2.54 (t, J=6.97 Hz, 2H) 3.31 (s, 3H) 3.51-3.59 (m, 2H) 7.18-7.28 (m, 3H) 7.48-7.66 (m, 3H) 7.72-7.93 (m, 14H). m/z 363 [M]⁺.

In a 250 mL round-bottomed flask in a dry ice bath sealed with a rubber septum and under argon was added (4-methoxy-4-oxobutyl)triphenylphosphonium bromide (10.66 g, 24.04 mmol) in THF (60 mL) to give a white suspension. It was held at −20° C. for a few minutes to equilibrate. NaHMDS (12.0 mL, 24.00 mmol) was added dropwise by syringe under argon and was stirred for 20 minutes after addition. The temperature was cooled to −65° C. A solution of 1 (4.71 g, 22.20 mmol) in 15 mL of dry THF and was added dropwise. The flask was allowed to rise to room temperature and stirred overnight. Saturated ammonium chloride solution was added to quench until the reaction changed color. It was extracted with ethyl acetate and more ammonium chloride solution, dried with Na₂SO₄, filtered, and evaporated. The crude material was purified by flash chromatography (5% ethyl acetate in hexanes) to give the product as an off-white solid (4.4 g, 61%). ¹H NMR (400 MHz, CDCl₃) δ ppm 2.36-2.45 (m, 2H) 2.61 (qd, J=7.42, 1.95 Hz, 2H) 3.65 (s, 3H) 5.11 (s, 3H) 5.59 (dt, J=11.72, 7.22 Hz, 1H) 6.41 (d, J=11.72 Hz, 1H) 7.21-7.26 (m, 3H) 7.33-7.50 (m, 14H). m/z 297 [M+H]⁺.

### Synthesis of Compound 3

In a 250 mL round-bottomed flask was added 2 (1.139 g, 3.84 mmol) and lithium hydroxide monohydrate (0.808 g, 19.25 mmol) in THF (20 mL) and water (20 mL) to give a white suspension. The reaction was stirred at room temperature for two days. The mixture was then treated with water (50 mL) and dichloromethane (100 mL) and the layers were allowed to stir overnight. The layers were separated, and the organic layer was extracted an additional time with water. The combined aqueous layers were acidified until it became milky white, then extracted with dichloromethane, dried with Na₂SO₄, filtered, and evaporated to yield 0.7217 g of 3 (2.56 mmol, 66% yield). ¹H NMR (500 MHz, CDCl₃) δ ppm 2.44-2.52 (m, 2H) 2.61-2.71 (m, 2H) 5.09 (s, 2H) 5.58-5.70 (m, 1H) 6.46 (d, J=11.74 Hz, 1H) 6.85-6.91 (m, 2H) 7.24-7.27 (m, 2H) 7.30-7.48 (m, 5H). m/z 283 [M+H]⁺.

### Synthesis of Compound 4

In a 200 mL reaction vial was added quinuclidin-3-ol (0.047 g, 0.370 mmol), N-bromosuccinimide (0.641 g, 3.60 mmol), and 3,4,5-trimethoxybenzoic acid (0.759 g, 3.58 mmol) in toluene (120 mL) to give a colorless solution. The solution was cooled to −20° C. for a few minutes. A solution of 3 (0.999 g, 3.54 mmol) in 20 mL of toluene was added dropwise to the flask. It was allowed to warm to room temperature and stirred overnight sealed. Toluene (85 mL) and 175 mL of 1 M HCl were added to quench reaction. The layers were separated and the organic layer was washed with 175 mL of a 1 M NaOH solution, then 175 mL of brine. The organic layer was dried with sodium sulfate, filtered, dried, and evaporated. Purification by flash chromatography using a gradient of ethyl acetate in hexanes gave 4 as a white solid (0.9525 g, 2.64 mmol, 74% yield). ¹H NMR (400 MHz, CDCl₃) δ ppm 1.90-2.26 (m, 2H) 2.29-2.51 (m, 2H) 4.81-4.89 (m, 1H) 4.91-4.96 (m, 1H) 5.07 (s, 2H) 6.91-7.09 (m, 3H) 7.26-7.45 (m, 6H). m/z 378 [M+H₂O]⁺.

### Synthesis of Compound 5

In a 100 mL round-bottomed flask was added 4 (0.638 g, 1.766 mmol) and 1,1,1,3,3,3-hexamethyl-2-(trimethylsilyl)trisilane (1.090 mL, 3.53 mmol) in toluene (35 mL) to give a colorless solution. The solution was heated to reflux, then AIBN (0.035 g, 0.212 mmol) was added and the reaction was maintained at reflux for 8 hours, then allowed to cool to room temperature overnight. The solvent was evaporated on a rotary evaporator and purified by flash chromatography using a gradient of ethyl acetate in hexanes gave 5 as a pale yellow solid (0.34 g, 1.2 mmol, 68% yield). ¹H NMR (400 MHz, CDCl₃) δ ppm 1.85-1.98 (m, 1H) 2.21 (dddd, J=12.84, 9.62, 6.83, 4.69 Hz, 1H) 2.30-2.52 (m, 2H) 2.87 (dd, J=14.06, 6.25 Hz, 1H) 3.04 (dd, J=14.06, 5.86 Hz, 1H) 4.71 (dt, J=13.67, 6.44 Hz, 1H) 5.04 (s, 2H) 6.78-6.91 (m, 3H) 7.21 (d, J=7.81 Hz, 1H) 7.26-7.46 (m, 5H). m/z 283 [M+H]⁺.

### Synthesis of Compound 6

In a 100 mL round-bottomed flask was added 5 (0.248 g, 0.878 mmol) in methanol (15 mL) to give a colorless solution. The air was purged by vacuum and filled with argon. A catalytic amount of 10% palladium on activated carbon was added. The argon was purged from the flask and refilled with hydrogen gas from a balloon. It was left stirring overnight. The solution was filtered on a bed of Celite and rinsed with methanol. The methanol was removed on a rotary evaporator and the residue was purified by flash chromatography (30% ethyl acetate in hexanes) to give 6 as a pale yellow solid (0.153 g, 0.8 mmol, 91% yield). ¹H NMR (500 MHz, CDCl₃) δ ppm 1.91-2.02 (m, 1H) 2.23-2.32 (m, 1H) 2.34-2.55 (m, 2H) 2.86-2.94 (m, 1H) 3.02 (dd, J=13.94, 6.11 Hz, 1H) 4.69-4.78 (m, 1H) 6.71-6.77 (m, 2H) 6.80 (d, J=7.83 Hz, 1H) 7.16-7.23 (m, 1H). m/z 193 [M+H]⁺.

### Synthesis of Compound 7

In a 100 mL round-bottomed flask was added 6 (0.324 g, 1.684 mmol), triethylamine (0.282 mL, 2.020 mmol), and DMAP (0.207 g, 1.693 mmol) in dry THF (15 mL) to give a colorless solution. A solution of 2,2,2-trichloroethyl sulfochloridate (0.449 g, 1.812 mmol) in dry THF (10 mL) was then added dropwise. After addition, a white solid precipitated. The mixture was left to stir overnight. The solution was extracted with 100 mL of ethyl acetate and washed with 50 mL of water, then with 2×50 mL of 0.5 M HCl, then with water, and last washed with saturated ammonium chloride. The organic layer was dried with sodium sulfate and evaporated. Purification by flash chromatography using a gradient of ethyl acetate in hexanes gave 7 as a clear oil (0.3132 g, 0.776 mmol, 46% yield). ¹H NMR (500 MHz, CDCl₃) δ ppm 1.89-2.01 (m, 1H) 2.42-2.59 (m, 2H) 2.97-3.04 (m, 1H) 3.04-3.12 (m, 1H) 4.68-4.77 (m, 1H) 4.86 (s, 2H) 7.24-7.31 (m, 3H) 7.38-7.44 (m, 1H). m/z 404 [M+H]⁺.

### Synthesis of Compound M36

In a 50 mL round-bottomed flask was added 7 (0.313 g, 0.776 mmol) in methanol (6 mL) to give a colorless solution. Ammonium formate (0.306 g, 4.85 mmol) and 10% palladium on activated carbon (0.675 g, 0.634 mmol) were added to give a clear suspension that was stirred at room temperature overnight. The solution was filtered on a bed of Celite and rinsed with methanol, which was concentrated on a rotary evaporator to give a crude oil. Purification by flash chromatography using 10:2:0.5 dichloromethane:methanol:ammonium hydroxide. Fractions containing the product were concentrated and the remaining aqueous residue was frozen and dried on a lyophilizer to give M36 as a gummy solid (0.2025 g, 0.70 mmol, 90% yield). ¹H NMR (500 MHz, DMSO-d₆) δ ppm 1.95-2.12 (m, 1H) 2.30-2.41 (m, 1H) 2.53-2.64 (m, 2H) 2.96-3.16 (m, 2H) 4.74-4.92 (m, 1H) 7.10 (br d, J=7.34 Hz, 1H) 7.20-7.30 (m, 6H) 7.33-7.40 (m, 1H). m/z 273 [M+H]⁺.

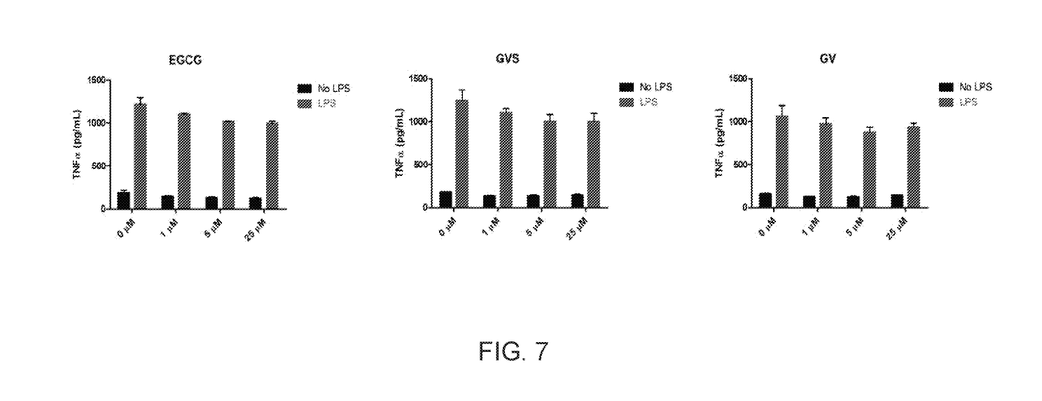

Graphs showing the effectiveness of some catechin derivatives in reducing TNF-α in presence of LPS are presented in FIG. 7.

Embodiments of the present disclosure further relate to any one or more of the following paragraphs:

A method of for making a catechin derivative, or a salt thereof, the method comprising:contacting an alcohol protected aromatic aldehyde and a triphenyl phosphonium ylide to make an alcohol protected aromatic olefin; hydrogenating the alcohol protected aromatic olefin to make alcohol protected aromatic compound; and deprotecting the alcohol protected aromatic compound to make the catechin derivative.

2. The method according to paragraph 1, wherein the contacting an alcohol protected aromatic aldehyde and a triphenyl phosphonium ylide to make an alcohol protected aromatic olefin comprises:

contacting a

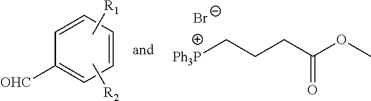

to make

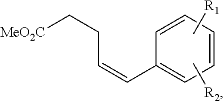

wherein wherein R₁ and R₂ are independently selected from the group consisting of —H, —OH, —OCH₃, —OBn, —OSO₃H, tert-butyldimethylsilyl ether, trimethylsilyl, tert-butyldiphenylsilyl, and triisopropylsilyl.

3. The method according to paragraph 1 or 2, wherein the hydrogenating the alcohol protected aromatic olefin to make alcohol protected aromatic compound comprises:

contacting

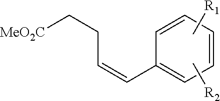

with a first base to make

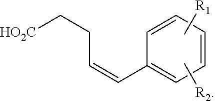

4. The method according to any one of paragraphs 1 to 3, wherein deprotecting the alcohol protected alcohol protected aromatic compound to make the catechin derivative comprises:

contacting

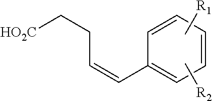

and N-bromosuccinimide to make

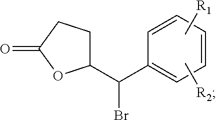

contacting

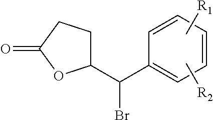

and a free radical initiator and a hydrogen donating compound to make

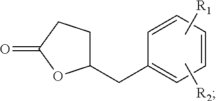

and
contacting

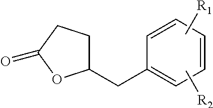

and a first hydrogenation catalyst to make the catechin derivative, wherein the catechin derivative has a formula

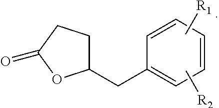

5. The method according to any one of paragraphs 1 to 4, wherein the base is selected from the group consisting of: sodium hydroxide, ammonium hydroxide, ammonium sulfate, lithium hydroxide, potassium hydroxide, triethylene tetraamine, diethylene triamine, triethylamine, urea, lithium diisopropylamide,

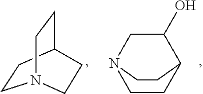

and mixtures thereof.

6. The method according to any one paragraphs 1 to 5, wherein the contacting

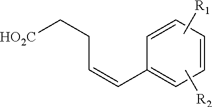

and N-bromosuccinimide to make

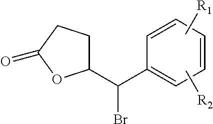

further comprises PhCO₂H, a second base or a chiral catalyst.

7. The method according to any one of paragraphs 1 to 6, wherein the second base is present and is selected from the group consisting of: sodium hydroxide, ammonium hydroxide, ammonium sulfate, lithium hydroxide, potassium hydroxide, triethylene tetraamine, diethylene triamine, triethylamine, urea, lithium diisopropylamide,

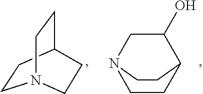

and mixtures thereof.

8. The method according to any one of paragraphs 1 to 7, wherein the chiral catalyst is present and is selected from (DHQ)₂PHAL and (DHQD)₂PHAL.

9. The method according to any one of paragraphs 1 to 8, wherein the free radical initiator is selected from a group consisting of: dicumyl peroxide, di-t-butyl peroxide, t-butylperoxybenzoate, 2,5-dimethyl-2,5-di(t-butylperoxy)hexane, t-butyl peroxyneodecanoate, 2,5-dimethyl-2,5-di(t-butylperoxy)hexyne, t-amyl peroxypivalate, 1,3-bis(t-butylperoxyisopropyl)benzene, tert-amylperoxy 2-ethyl hexanoate, t-butylperoxy 2-ethyl hexanoate, t-butyl peroxy isobutyrate, t-butylperoxy isopropyl carbonate, t-butylperoxy 3,5,5-trimethylhexanoate, 2,5-dimethyl-2,5-di(benzolyperoxy)hexane, n-butyl 4,4-di(t-butylperoxy)valcratic, t-butylcumyl peroxide, di(2-t-butylperoxy isopropyl)benzene, t-butyl hydroperoxide, cumyl hydroperoxide and mixtures thereof. Examples of suitable azo compounds include 2,2′-azobisisopropionitrile, 2,2′-azobisisobutyronitrile (AIBN), dimethyl azoisobutyrate, 1,1′-azobis (cyclohexanecarbonitrile), and 2,2′-azobis(2-methylpropane).

10. The method according to any one of paragraphs 1 to 9, wherein the first hydrogenation catalyst is selected from the group consisting of: platinum, palladium, Pd/C, rhodium, ruthenium and nickel.

11. The method according to any one of paragraphs 1 to 10, wherein the first hydrogen donating compound is selected from the group consisting of: H₂, formic acid, isopropanol, and dihydroanthracene.

12. The method according to any one of paragraphs 1 to 11, wherein the method further comprises:

contacting the catechin derivative,

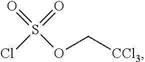

and a third base to make

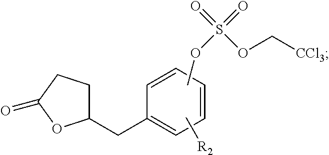

contacting

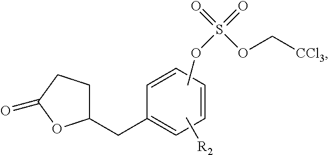

a second hydrogenation catalyst, and second hydrogen donating compound to make

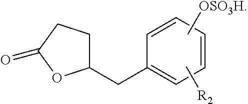

13. The method according to any one paragraphs 1 to 12, wherein the third base is selected from the group consisting of: is selected from the group consisting of: sodium hydroxide, ammonium hydroxide, ammonium sulfate, lithium hydroxide, potassium hydroxide, triethylene tetraamine, diethylene triamine, triethylamine, urea, lithium diisopropylamide,

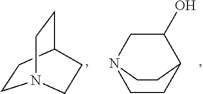

and mixtures thereof.

14. The method according to any of one paragraphs 1 to 13, wherein the second hydrogenation catalyst is selected from the group consisting of: platinum, palladium Pd/C, rhodium, ruthenium and nickel.

15. The method according to any one of paragraphs 1 to 14, wherein the second hydrogen donating compound is selected from the group consisting of: H₂, formic acid, isopropanol, and dihydroanthracene.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

### REFERENCES

1. Liang, J.; Xu, F,; Zhang, Y.; Zang, X.; Wang, D.; Shang, M.; Wang, X.; Chui, D.; Cai, S. Biomedical Chromatography, 2013, DOI 10.1002/bmc.3034
2. Kurlbaum, M.; Mulek, M.; Hogger, P. Plos One, 2013, 8, 1-10
3. von Oettingen, W. F., Condensation Products of Aromatic Aldehydes with Δ₂-Angelica Lactone. J Am Chem Soc., 1930, 52(5) pp. 2024-2025
4. McKillop, et al., Further Functional Group Oxidations Using Sodium Perborate, Tetrahedron, 1989, 45(11) pp. 3299-3306
5. Talukdar, et al., VO(AcAc)₂: An Efficient Catalyst for the Oxidation of Aldehydes to the Corresponding Acids in the Presence of Aqueous H₂O₂. Synlett. 2013; 24(8):963-66).

We claim:

1. A method for making a catechin derivative, or a salt thereof, the method comprising:
contacting an alcohol protected aromatic aldehyde and a triphenyl phosphonium ylide to make an alcohol protected aromatic olefin;
hydrogenating the alcohol protected aromatic olefin to make an alcohol protected aromatic compound; and
deprotecting the alcohol protected aromatic compound to make the catechin derivative.

2. The method of claim 1, wherein the contacting an alcohol protected aromatic aldehyde and a triphenyl phosphonium ylide to make an alcohol protected aromatic olefin comprises:
contacting a 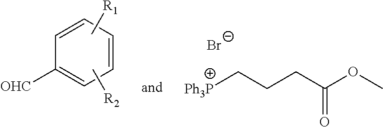
to make 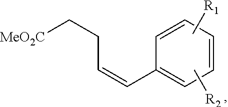
wherein wherein R₁ and R₂ are independently selected from the group consisting of —H, —OH, —OCH₃, —OBn, —OSO₃H, tert-butyldimethylsilyl ether, trimethylsilyl, tert-butyldiphenylsilyl, and triisopropylsilyl.

3. The method of claim 2, wherein the hydrogenating the alcohol protected aromatic olefin to make alcohol protected aromatic compound comprises:
contacting 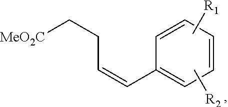
with a first base to make

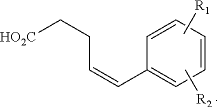

4. The method of claim 3, wherein deprotecting the alcohol protected alcohol protected aromatic compound to make the catechin derivative comprises:
contacting 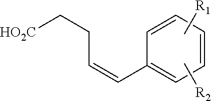
and N-bromosuccinimide to make 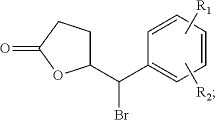
contacting 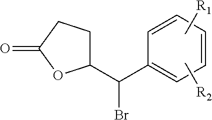
and a free radical initiator and a hydrogen donating compound to make 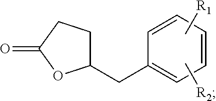
and
contacting 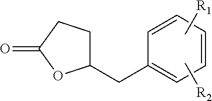
and a first hydrogenation catalyst to make the catechin derivative, wherein the catechin derivative has a formula

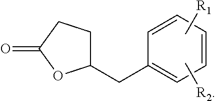

5. The method of claim 4, wherein the base is selected from the group consisting of: sodium hydroxide, ammonium hydroxide, ammonium sulfate, lithium hydroxide, potassium hydroxide, triethylene tetraamine, diethylene triamine, triethylamine, urea, lithium diisopropylamide, 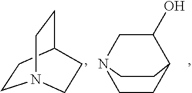
and mixtures thereof.

6. The method of claim 5, wherein the contacting 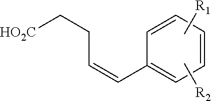
and N-bromosuccinimide to make 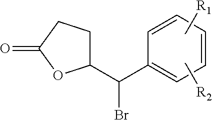
further comprises PhCO₂H, a second base or a chiral catalyst.

7. The method of claim 6, wherein the second base is present and is selected from the group consisting of: sodium hydroxide, ammonium hydroxide, ammonium sulfate, lithium hydroxide, potassium hydroxide, triethylene tetraamine, diethylene triamine, triethylamine, urea lithium diisopropylamide, 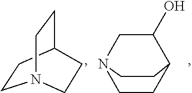
and mixtures thereof.

8. The method of claim 6, wherein the chiral catalyst is present and is selected from (DHQ)₂PHAL and (DHQD)₂PHAL.

9. The method of claim 6, wherein the free radical initiator is selected from a group consisting of: dicumyl peroxide, di-t-butyl peroxide, t-butylperoxybenzoate, 2,5-dimethyl-2,5-di(t-butylperoxy)hexane, t-butyl peroxyneodecanoate, 2,5-dimethyl-2,5-di(t-butylperoxy)hexyne, t-amyl peroxypivalate, 1,3-bis(t-butylperoxyisopropyl)benzene, tert-amylperoxy 2-ethyl hexanoate, t-butylperoxy 2-ethyl hexanoate, t-butyl peroxy isobutyrate, t-butylperoxy isopropyl carbonate, t-butylperoxy 3,5,5-trimethylhexanoate, 2,5-dimethyl-2,5-di(benzolyperoxy)hexane, n-butyl 4,4-di(t-butylperoxy)valcratic, t-butylcumyl peroxide, di(2-t-butylperoxy isopropyl)benzene, t-butyl hydroperoxide, cumyl hydroperoxide and mixtures thereof. Examples of suitable azo compounds include 2,2′-azobisisopropionitrile, 2,2′-azobisisobutyronitrile (AIBN), dimethyl azoisobutyrate, 1,1′-azobis (cyclohexanecarbonitrile), and 2,2′-azobis(2-methylpropane).

10. The method of claim 6, wherein the first hydrogenation catalyst is selected from the group consisting of: platinum, palladium, Pd/C, rhodium, ruthenium, and nickel.

11. The method of claim 6, wherein the first hydrogen donating compound is selected from the group consisting of: H₂, formic acid, isopropanol, and dihydroanthracene.

12. The method of claim 1, further comprising:
contacting the catechin derivative, 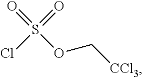
and a third base to make 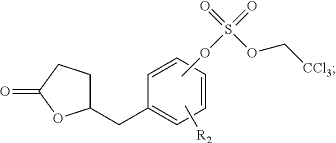
contacting 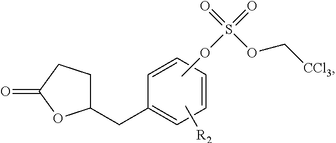
a second hydrogenation catalyst, and second hydrogen donating compound to make

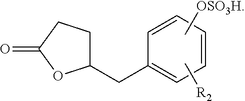

13. The method of claim 12, wherein the third base is selected from the group consisting of: is selected from the group consisting of: sodium hydroxide, ammonium hydroxide, ammonium, sulfate, lithium hydroxide, potassium hydroxide, triethylene tetraamine, diethylene triamine, triethylamine, urea, lithium diisopropylamide, 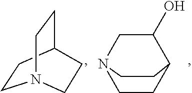
and mixtures thereof.

14. The method of claim 13, wherein the second hydrogenation catalyst is selected from the group consisting of: platinum, palladium, Pd/C, rhodium, ruthenium, and nickel.

15. The method of claim 14, wherein the second hydrogen donating compound is selected from the group consisting of: H₂, formic acid, isopropanol, and dihydroanthracene.
